(19) United States
(12) Patent Application Publication
(10) Pub. No.: US 2014/0005493 A1
(43) Pub. Date: Jan. 2, 2014

(54) APPARATUS AND METHOD FOR DETECTING MISALIGNMENT BETWEEN IMPLANTABLE BIO SENSOR AND BIO-SIGNAL RECEIVING MODULE

(71) Applicant: Samsung Electronics Co. Ltd., Suwon-si (KR)

(72) Inventors: Do-Young KIM, Hwaseong-si (KR); Jeong-Je PARK, Hwaseong-si (KR); Kwang-Hyeon LEE, Seoul (KR); Nae-Hyun LIM, Seoul (KR); Dong-Keon KONG, Suwon-si (KR); Yong CHANG, Seongnam-si (KR)

(21) Appl. No.: 13/933,516

(22) Filed: Jul. 2, 2013

(30) Foreign Application Priority Data
Jul. 2, 2012 (KR) 10-2012-0071873

(51) Int. Cl. A61B 5/00

(52) U.S. Cl. CPC A61B 5/7221
USPC 600/300

(57) ABSTRACT

A method for detecting in a bio sensor misalignment between the bio sensor and a bio signal receiving module is provided. The method includes receiving, by the bio sensor, a light signal from the bio signal receiving module, determining, by the bio sensor, whether the light signal is received in a misalignment detection section, determining, by the bio sensor, that the bio sensor and the bio signal receiving module are misaligned if the light signal is received in the misalignment detection section, generating a feedback signal that comprises information to indicate misalignment between the bio sensor and the bio signal receiving module, and transmitting, by the bio sensor, the feedback signal to the bio signal receiving module.

### CROSS-REFERENCE TO RELATED APPLICATION(S)

This application claims the benefit under 35 U.S.C. §119(a) of a Korean patent application filed on Jul. 2, 2012 in the Korean Intellectual Property Office and assigned Serial No. 10-2012-0071873, the entire disclosure of which is incorporated hereby incorporated by reference.

### TECHNICAL FIELD

The present disclosure relates to implantable bio sensors. More particularly, the present disclosure relates to an apparatus and method for detecting misalignment between an implantable bio sensor and a bio signal receiving module.

### BACKGROUND

Bio sensors refer to devices for examining characteristics of an object using functions known to exist in living creatures. Bio sensors are used as detectors for life forms because bio sensors have superior sensitivity and reaction specificity. Such bio sensors are widely used in medicine and medical fields. In addition, uses for bio sensors are continuing to expand. In particular, medical diagnostics uses a large number of bio sensors to analyze biometric samples including blood.

Bio sensors are involved in enzymatic analysis and immunoassay depending on types of detectors and are classified into optical bio sensors and electrochemical bio sensors depending on methods for quantitative analysis on an object in the biometric sample.

Optical bio sensors are most commonly used to measure a density of an object by measuring light transmittance, absorbance, or a change in wavelength, which has an advantage of having less deviation over measuring time because reaction mechanisms of various objects for analysis have been already known to the public and measurement is made after reaction has been made for sufficient time. In addition, optical bio sensors may be implanted into life forms and may transmit bio signals that represent quantitative analysis results of the biometric sample to a bio signal receiving module (e.g., a wrist watch, a patch, a band, or the like) using visual light.

However, if the bio signal receiving module is located outside of the life form, the location of the bio signal receiving module may be changed due to movements of the life form or due to external forces. Deviation in the location of the receiving module leads to misalignment between the bio sensor and the receiving module, which in turn may result in the bio sensor being unable to communicate with the bio signal receiving module or error in transmission or reception of the bio signal. In addition, visible light to be used in transmission/reception of the bio signal may be scattered due to moisture, such as perspiration, which in turn causes an error in transmission/reception.

Therefore, a need exists to address the impossibility or error in transmitting or receiving bio signals.

The above information is presented as background information only to assist with an understanding of the present disclosure. No determination has been made, and no assertion is made, as to whether any of the above might be applicable as prior art with regard to the present disclosure.

### SUMMARY

Aspects of the present disclosure are to address at least the above-mentioned problems and/or disadvantages and to provide at least the advantages described below. Accordingly, an aspect of the present disclosure is to provide an apparatus and method for detecting misalignment between an implantable bio sensor and a bio sensor receiving module.

In accordance with an aspect of the present disclosure, a method for detecting, in a bio sensor, misalignment between the bio sensor and a bio signal receiving module is provided. The method includes receiving, by the bio sensor, a light signal from the bio signal receiving module, determining, by the bio sensor, whether the light signal is received in a misalignment detection section, determining, by the bio sensor, that the bio sensor and the bio signal receiving module are misaligned if the light signal is received in the misalignment detection section, and generating a feedback signal that comprises information to indicate misalignment between the bio sensor and the bio signal receiving module, and transmitting, by the bio sensor, the feedback signal to the bio signal receiving module.

In accordance with another aspect of the present disclosure, a method for detecting, in a bio signal receiving module, misalignment between a bio sensor and the bio signal receiving module is provided. The method includes transmitting, by the bio signal receiving module, a light signal to the bio sensor, determining, by the bio signal receiving module, whether a bio signal from the bio sensor is received in a misalignment detection section, and determining, by the bio signal receiving module, that the bio signal receiving module and the bio sensor are misaligned if the bio signal is received in the misalignment detection section.

In accordance with another aspect of the present disclosure, a bio sensor for detecting misalignment between the bio sensor and a bio signal receiving module is provided. The bio sensor includes a photovoltaic power source configured to receive a light signal from the bio signal receiving module, a detector configured to determine whether the light signal is received in a misalignment detection section, and to determine that the bio sensor and the bio signal receiving module are misaligned if the light signal is received in the misalignment detection section, and a transmitter to generate a feedback signal that comprises information to indicate misalignment between the bio sensor and the bio signal receiving module and to transmit the feedback signal to the bio signal receiving module.

In accordance with another aspect of the present disclosure, a bio signal receiving module for detecting misalignment between a bio sensor and the bio signal receiving module is provided. The bio signal receiving module includes a transmitter configured to transmit a light signal to the bio sensor, and a detector configured to determine whether a bio signal from the bio sensor is received in a misalignment detection section, and to determine that the bio signal receiving module and the bio sensor are misaligned if the bio signal is received in the misalignment detection section.

Other aspects, advantages, and salient features of the disclosure will become apparent to those skilled in the art from the following detailed description, which, taken in conjunction with the annexed drawings, discloses various embodiments of the present disclosure.

### BRIEF DESCRIPTION OF THE DRAWINGS

The above and other aspects, features, and advantages certain embodiments of the present disclosure will become more apparent from the following description taken in conjunction with the accompanying drawings in which:

FIGS. 1A to 1C illustrate transmission and reception of bio signals between a bio sensor and a bio signal receiving module according to an embodiment of the present disclosure;

FIG. 2 illustrates detection of misalignment between a receiving module and a bio sensor according to an embodiment of the present disclosure;

FIG. 3 illustrates detection of misalignment between a receiving module and a bio sensor according to another embodiment of the present disclosure;

FIG. 4 illustrates detection of misalignment between a receiving module and a bio sensor according to another embodiment of the present disclosure;

FIG. 5 is a flowchart illustrating a method for a bio sensor to detect misalignment with a bio signal receiving module according to an embodiment of the present disclosure; and

FIG. 6 is a flowchart illustrating a method for a bio signal receiving module to detect misalignment with a bio sensor according to an embodiment of the present disclosure.

Throughout the drawings, like reference numerals will be understood to refer to like parts, components, and structures.

### DETAILED DESCRIPTION

The following description with reference to the accompanying drawings is provided to assist in a comprehensive understanding of various embodiments of the present disclosure as defined by the claims and their equivalents. It includes various specific details to assist in that understanding, but these are to be regarded as merely exemplary. Accordingly, those of ordinary skill in the art will recognize that various changes and modifications of the various embodiments described herein can be made without departing from the scope and spirit of the present disclosure. In addition, descriptions of well-known functions and constructions may be omitted for clarity and conciseness.

The terms and words used in the following description and claims are not limited to the bibliographical meanings, but are merely used by the inventor to enable a clear and consistent understanding of the present disclosure. Accordingly, it should be apparent to those skilled in the art that the following description of various embodiments of the present disclosure is provided for illustration purposes only and not for the purpose of limiting the present disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

According to various embodiments, a receiving module may include a transmitter. The transmitter may be a Light Emitting Diode (LED), and/or the like.

According to various embodiments, the receiving module may include a power source. The power source may include LEDs, and/or the like.

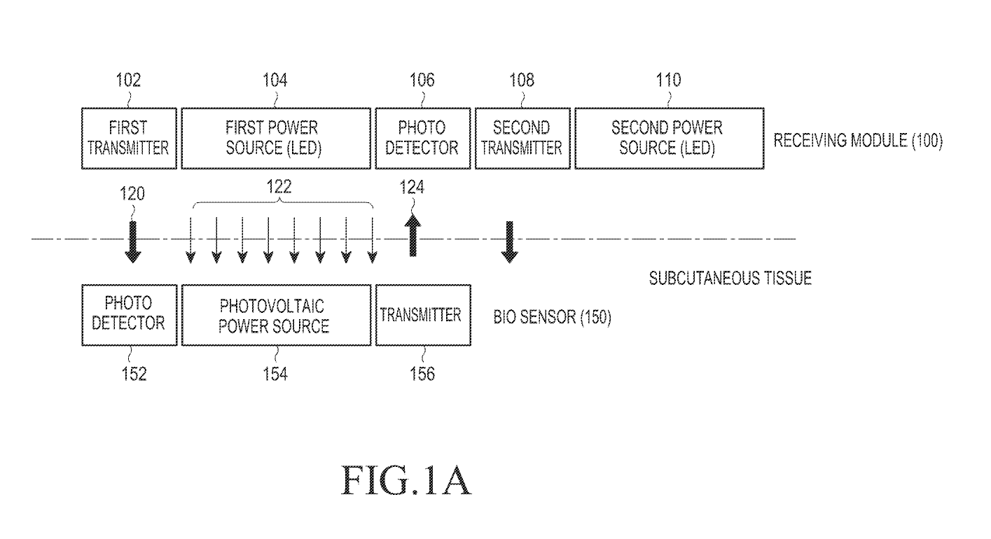

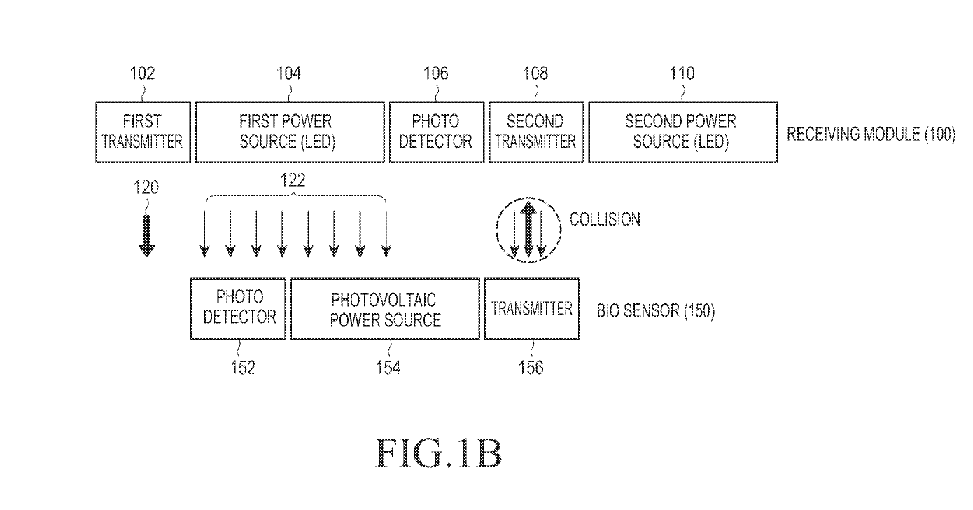

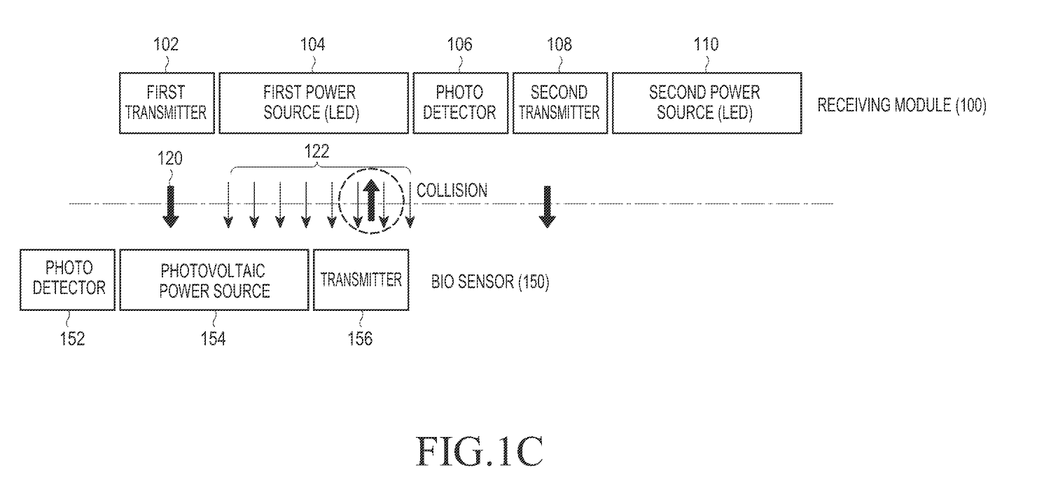

FIGS. 1A to 1C illustrate transmission and reception of bio signals between a bio sensor and a bio signal receiving module according to an embodiment of the present disclosure.

Referring to FIGS. 1A to 1C, the bio sensor is assumed herein to refer to an optical bio sensor that uses visible light.

A bio sensor 150 which is implanted into subcutaneous tissues of a life form includes a photo detector 152, a photovoltaic power source 154, and a transmitter 156. The bio sensor 150 transmits bio signals that represent quantitative analysis results of a biometric sample.

A receiving module 100 (also referred to as bio signal receiving module) to receive bio signals is located outside of the life form and includes a first transmitter 102, a first power source 104, a photo detector 106, a second transmitter 108, and a second power source 110.

According to various embodiments, the first power source 104 and the second power source 110 may be Light Emitting Diodes (LEDs).

The receiving module 100 may correctly receive bio signals when the first transmitter 102, the first power source 104, and the photo detector 106 correspond one to one with the photo detector 152, the photovoltaic power source 154, and the transmitter 156 of the bio sensor 150. As illustrated in FIG. 1A, the first transmitter 102, the first power source 104, and the photo detector 106 of the receiving module 100 correspond one to one with the photo detector 152, the photovoltaic power source 154, and the transmitter 156 of the bio sensor 150.

The first transmitter 102 of the receiving module 100 transmits a ready signal 120 to notify that the first power source 104 will transmit light signals. The first power source 104 transmits light signals 122. The ready signal 120 and light signals 122 may be transmitted at certain intervals based on the configuration of the receiving module 100 and/or the bio sensor 150.

The photo detector 152 of the bio sensor 150 receives the ready signal from the first transmitter 102 of the receiving module 100, and the photovoltaic power source 154 receives light signals from the first power source 104 of the receiving module 100. The transmitter 156 of the bio sensor 150 obtains from the received light signals 122 (e.g., through power supplied by the photovoltaic power source 154) energy for transmission of bio signals, and transmits bio signals 124 using the obtained energy.

The photo detector 106 of the receiving module 100 receives bio signals 124 from the transmitter 156 of the bio sensor 150, analyzes the bio signals 124, and then transmits the analysis result in Radio Frequency (RF) waves.

As such, the bio sensor 150 and the receiving module 100 may exchange bio signals in the way as described above. However, because the receiving module 100 is located outside of the life form, the location of the receiving module 100 may be changed due to a movement of the life form and/or due to external forces applied to the receiving module 100, in which case the correct exchange of bio signals between the bio sensor 150 and the receiving module 100 becomes difficult.

For example, as illustrated in FIG. 1B, when the receiving module 100 is deviated (e.g., moved) to the left of the bio sensor 150, the receiving module 100 has difficulty correctly receiving bio signals because bio signals transmitted from the transmitter 156 of the bio sensor 150 collide with a ready signal transmitted from the second transmitter 108 of the receiving module 100.

As another example, as illustrated in FIG. 1C, when the receiving module 100 is deviated (e.g., moved) to the right of the bio sensor 150, the receiving module 100 has difficulty correctly receiving bio signals because bio signals transmitted from the transmitter 156 of the bio sensor 150 collide with light signals transmitted from the first power source 104 of the receiving module 100.

FIGS. 1B and 1C illustrate that, even though the receiving module 100 is deviated, the photovoltaic power source 154 of the bio sensor 150 is enabled to transmit (e.g., generate power to enable transmission of) bio signals by receiving a portion of the light signals from the first power source 104 of the receiving module 100. However, the bio sensor 150 is a passive device that may not transmit bio signals at all if the photovoltaic power source 154 of the bio sensor 150 does not receive any light signals from the first power source 104 of the receiving module 100.

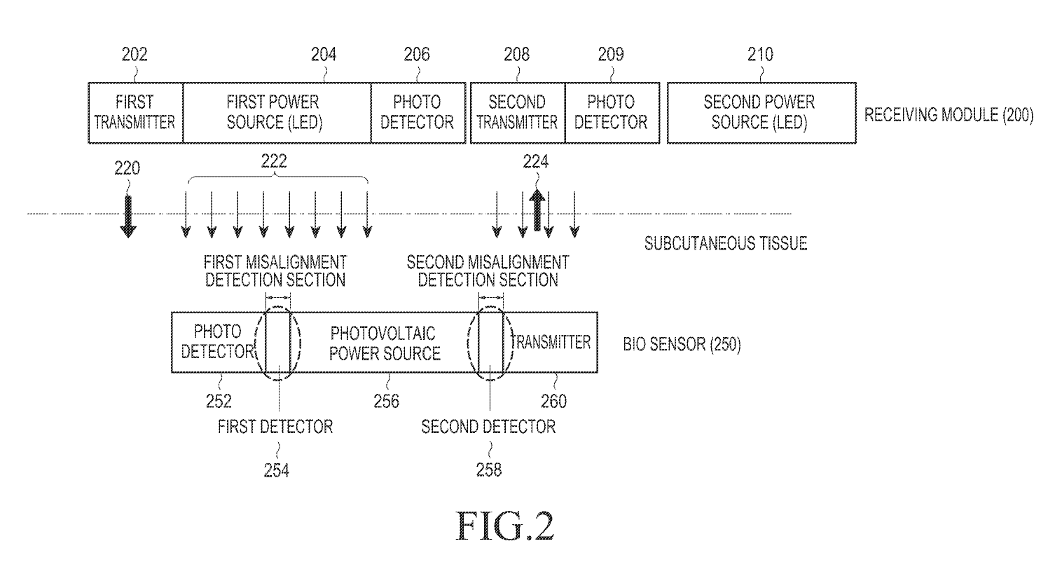

In this regard, a method for detecting deviation of the receiving module 100 (e.g., a misalignment between the receiving module 100 and the bio sensor 150), to correctly exchange bio signals will be described in connection with FIGS. 2 to 4.

FIG. 2 illustrates detection of misalignment between a receiving module and a bio sensor according to an embodiment of the present disclosure.

According to various embodiments, if the receiving module is deviated to the left of the bio sensor, the bio sensor detects the misalignment between the receiving module and the bio sensor and feeds the detection result back to the receiving module.

Referring to FIG. 2, the bio sensor 250 which is implanted in subcutaneous tissues of a life form includes a photo detector 252, a first detector 254, a photovoltaic power source 256, a second detector 258, and a transmitter 260 to transmit bio signals. The first detector 254 and the second detector 258 detect misalignment between the bio sensor 250 and the receiving module 200. For example, the first detector 254 detects the receiving module 200 being deviated to the left of the bio sensor 250 and the second detector 258 detects the receiving module 200 being deviated to the right of the bio sensor 250. As illustrated, the first detector 254 is disposed in an area referred to as a first misalignment detection section, and the second detector 258 is disposed in an area referred to as a second misalignment detection section.

As illustrated in FIG. 2, the first detector 254 is located between the photo detector 252 and the photovoltaic power source 256, and the second detector 258 is located between the photovoltaic power source 256 and the transmitter 260. However, according to various embodiments, the first detector 254 and the second detector 258 may be located anywhere in which the bio sensor 250 may easily detect the deviation of the receiving module 200 from the bio sensor 250.

A receiving module 200 (also referred to as a bio signal receiving module) that receives bio signals is located outside of the life form and includes a first transmitter 202, a first power source 204, a photo detector 206, a second transmitter 208, a photo detector 209 and a second power source 210. According to various embodiments, the first power source 204 and the second power source 210 may be LEDs.

The first transmitter 202 of the receiving module 200 transmits a ready signal 220 to notify that the first power source 204 will transmit light signals, and the first power source 204 transmits light signals 222. Reception of the light signal (e.g., or portion of the light signals 222) by the photovoltaic power source 256 of the bio sensor 250 allows for correct reception of bio signals.

However, because the receiving module 200 of FIG. 2 is deviated to the left of the bio sensor 250, the first detector 254 also receives the light signal. Upon reception of the light signal, the first detector 254 of the bio sensor 250 determines that the receiving module 200 is deviated to the left of the bio sensor 250. The transmitter 260 of the bio sensor 250 transmits a feedback signal 224 to notify that the receiving module 200 is deviated to the left to the receiving module 200. The feedback signal only includes information to indicate left or right deviation. For example, the feedback signal may not include any bio signals. The photo detector 209 of the receiving module 200 receives the feedback signal and recognizes that the receiving module 200 is deviated to the left.

Furthermore, although not shown, if the receiving module 200 is deviated to the right of the bio sensor 250, the light signal 222 transmitted by the first power source 204 of the receiving module 200 is received by the second detector 258 of the bio sensor 250. Upon reception of the light signal, the second detector 258 determines that the receiving module 200 is deviated to the right of the bio sensor 250. The transmitter 260 of the bio sensor 250 transmits a feedback signal to notify that the receiving module 200 is deviated to the right to the receiving module 200. The photo detector 209 of the receiving module 200 receives the feedback signal and recognizes that the receiving module 200 is deviated to the right.

The feedback signal may be configured as a feedback message that includes control information as shown in Table 1 below.

TABLE 1

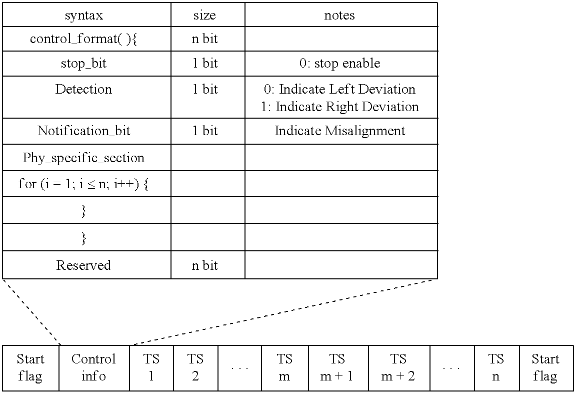

The control information shown in Table 1 includes Stop bit information, detection information, and Notification bit information.

The Stop_bit information, which may be represented with one bit, includes information that notifies the second transmitter 208 (e.g., the receiving module 200) to stop transmitting the ready signal in order to avoid interference (e.g., due to an overlay of the feedback signal transmitted by the transmitter 260 of the bio sensor 250 and the ready signal transmitted by the second transmitter 208 of the receiving module 200). The Stop_bit information may include information notifying the receiving module 200 of the reason for stopping transmission of the ready signal (e.g., so as to avoid interference between the ready signal transmitted by the second transmitter 208 and the feedback signal transmitted by the transmitter 260).

The detection information indicates in which direction the receiving module is deviated from the bio sensor. The direction in which the receiving module is deviated may be represented with 1 bit. For example, if the bit for the detection information is ‘0’, the direction information indicates that the receiving module is deviated to the left, and if the bit is ‘1’, the deviation information indicates that the receiving module is deviated to the right.

The Notification_bit information indicates a misalignment between the receiving module and the bio sensor. The indication of the misalignment between the receiving module and the bio sensor may be represented with 1 bit.

FIG. 2 illustrates that photo detector 206 and the photo detector 209 are located after (e.g., to the right of) the first power source 204 and the second transmitter 208, respectively. However, it will be obvious that the same functions as shown in FIG. 2, such as, for example, the photo detector 206 and the photo detector 209 detecting the feedback signal transmitted by the transmitter 260 of the bio sensor 250 may be implemented by applying a film of photo detector on the top of the receiving module 200 and vacating the spaces of the photo detectors 206 and 209. As an example, a film of photo detector may be applied to the entire top (e.g., surface) of the receiving module 250.

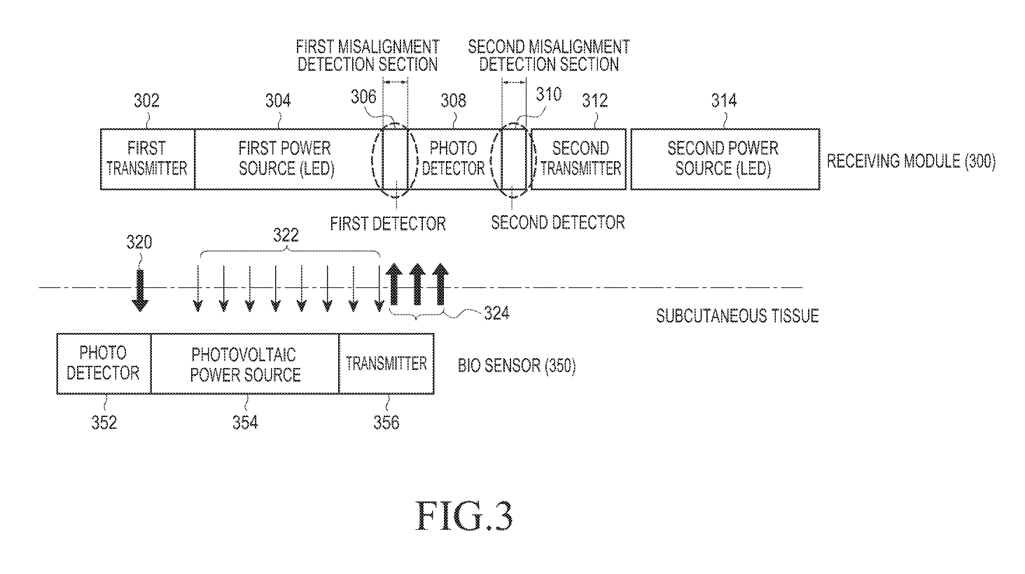

FIG. 3 illustrates detection of misalignment between a receiving module and a bio sensor according to another embodiment of the present disclosure.

According to various embodiments, the receiving module directly detects the alignment between the receiving module and the bio sensor. For example, assuming that the receiving module is deviated to the right of the bio sensor, the receiving module may directly detect the alignment or misalignment between the receiving module and the bio sensor.

Referring to FIG. 3, the bio sensor 350 which is implanted in subcutaneous tissues of a life form includes a photo detector 352, a photovoltaic power source 354, and a transmitter 356 and transmits bio signals.

A receiving module 300 (also referred to as a bio signal receiving module) to receive bio signals is located outside of the life form and includes a first transmitter 302, a first power source 304, a first detector 306, a photo detector 308, a second detector 310, a second transmitter 312, and a second power source 314. The first power source 304 and the second power source 314 may be LEDs. The first detector 306 and the second detector 310, individually or in combination, detects the receiving module 300 being deviated relative to the bio sensor. For example, if the receiving module 300 is deviated to the right of the bio sensor 350, the first detector 306 serves as a misalignment detector and detects the receiving module 300 being deviated to the right of the bio sensor 350. As another example, if the receiving module 300 is deviated to the left of the bio sensor 350, the second detector 310 serves as the misalignment detector 306 and detects the receiving module 300 being deviated to the left of the bio sensor 350.

As illustrated in FIG. 3, the first detector 306 is located between the first power source 304 and the photo detector 308 and the second detector 310 is located between the photo detector 308 and the second transmitter 312. However, according to various embodiments, the first detector 306 and the second detector 310 may be located anywhere for the receiving module 300 to easily detect the deviation of the receiving module 300 from the bio sensor 350.

The first transmitter 302 of the receiving module 300 transmits a ready signal 320 to notify that the first power source 304 will transmit light signals, and the first power source 304 transmits light signals 322. Reception of the light signals 322 (e.g., or a portion of the light signals 322) by the photovoltaic power source 354 of the bio sensor 350 allows for correct reception of bio signals 324. In addition, the photo detector 308 of the receiving module 300 should receive the bio signal from the bio sensor 350.

However, because the receiving module 300 of FIG. 3 is deviated to the right of the bio sensor 350, the bio signal 324 transmitted by the transmitter 356 of the bio sensor 350 is also received by the first detector 306 of the receiving module 300. Upon reception of the bio signal 324, the first detector 306 of the receiving module 300 determines that the receiving module 300 is deviated to the right of the bio sensor 350. For example, reception of the bio signal 324 by the first detector 306 may be indicative of a misalignment between the receiving module 300 and the bio sensor 350.

Furthermore, although not shown, if the receiving module 300 is deviated to the left of the bio sensor 350, the bio signal 324 transmitted by the transmitter 356 of the bio sensor 350 is received by the second detector 310 of the receiving module 300. Upon reception of the bio signal 324, the second detector 310 of the receiving module 300 detects and recognizes that the receiving module 300 is deviated to the left of the bio sensor 350.

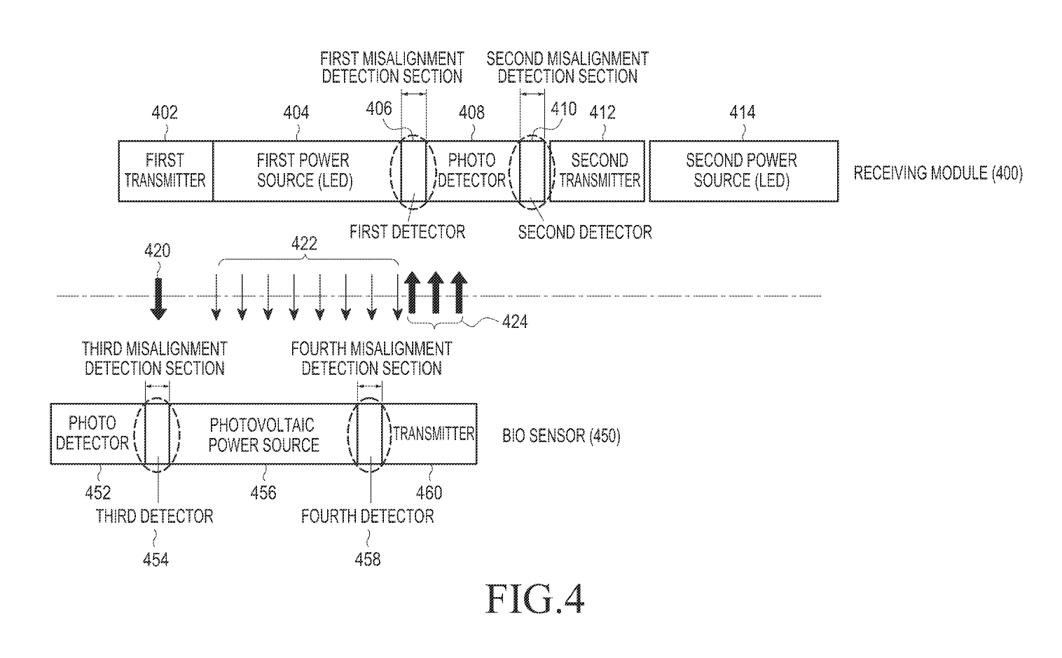

FIG. 4 illustrates detection of misalignment between a receiving module and a bio sensor according to another embodiment of the present disclosure.

According to various embodiments, the receiving module and the bio sensor each detect the alignment between the receiving module and the bio sensor.

Referring to FIG. 4, the receiving module is assumed to be deviated to the right of the bio sensor.

A bio sensor 450 which is implanted in subcutaneous tissues of a life form includes a photo detector 452, a third detector 454, a photovoltaic power source 456, a fourth detector 458, and a transmitter 460. The bio sensor 450 transmits bio signals. The third detector 454 and the fourth detector 458 detect misalignment between the bio sensor 450 and the receiving module 400. Specifically, the third detector 454 detects the receiving module 400 being deviated to the left of the bio sensor 450, and the fourth detector 458 detects the receiving module 400 being deviated to the right of the bio sensor 450. As described in connection with FIGS. 2 and 3, the third detector 454 and the fourth detector 458 may be located anywhere that allows for the bio sensor 450 to easily detect the deviation of the receiving module 400 relative to the bio sensor 450.

A receiving module 400 (also referred to as a bio signal receiving module) to receive bio signals is located outside of the life form. The receiving module 400 includes a first transmitter 402, a first power source 404, a first detector 406, a photo detector 408, a second detector 410, a second transmitter 412, and a second power source 414. The first power source 404 and the second power source 414 may be LEDs. The first detector 406 and the second detector 410, either individually or in combination, detect misalignment between the bio sensor 450 and the receiving module 400. Specifically, the first detector 406 detects the receiving module 400 being deviated to the right of the bio sensor 450, and the second detector 410 detects the receiving module 400 being deviated to the left of the bio sensor 450. As described in connection with FIGS. 2 and 3, the first detector 406 and the second detector 410 may be located anywhere that allows for the bio sensor 450 to easily detect the deviation of the receiving module 400 relative to the bio sensor 450.

The first transmitter 402 of the receiving module 400 transmits a ready signal 420 to notify that the first power source 404 is going to transmit light signals, and the first power source 404 transmits light signals 422. For correct reception of bio signals, the light signal has to be received only by the photovoltaic power source 456 of the bio sensor 450 and the bio signal transmitted from the transmitter 460 of the bio sensor 450 also has to be received by the photo detector 408 of the receiving module 400.

However, because the receiving module 400 of FIG. 4 is deviated to the right of the bio sensor 450, the fourth detector 458 also receives the light signal 422. Upon reception of the light signal 422, the fourth detector 458 of the bio sensor 450 determines that the receiving module 400 is deviated to the right of the bio sensor 450. As a result, the transmitter 460 of the bio sensor 450 transmits a feedback signal 424 to notify that the receiving module 400 is deviated relative to the receiving module. For example, the transmitter 460 of the bio sensor 450 transmits a feedback signal 424 to notify that the receiving module 400 is deviated to the right to the receiving module 400.

Upon reception of the feedback signal from the bio sensor 450, the first detector 450 of the receiving module 400 recognizes that the receiving module 400 is deviated to the right and re-confirms the recognized result by checking information contained in the feedback signal. According to such embodiments, detection reliability may be increased because the receiving module 400 and bio sensor 450 each detect misalignment between the receiving module 400 and the bio sensor 450.

Furthermore, although not shown, if the receiving module 400 is deviated to the left of the bio sensor 450, the light signal 422 transmitted by the first power source 404 of the receiving module 400 is received by the second detector 454 of the bio sensor 450. Upon reception of the light signal, the third detector 454 (e.g., the bio sensor 450) determines that the receiving module 400 is deviated to the right of the bio sensor 450, and the transmitter 460 of the bio sensor 250 transmits a feedback signal 424 to notify that the receiving module 400 is deviated to the left of the bio sensor 450.

Upon reception of the feedback signal from the bio sensor 450, the second detector 410 of the receiving module 400 recognizes that the receiving module 400 is deviated to the left and re-confirms the recognized result by checking (e.g., analyzing) information included in the feedback signal.

Although the bio sensor is illustrated to include the photo detector, the photovoltaic power source, and the transmitter arranged in the sequence as illustrated in FIGS. 1 to 4, the photo detector, the photovoltaic power source, and the transmitter may be arranged in any sequence (e.g., the bio sensor may be configured in any form), depending on cases. Furthermore, although the receiving module is illustrated to have the transmitter, the power source, and the photo detector arranged to correspond to the bio sensor in FIGS. 1 to 4, the receiving module may be configured in any form depending on cases.

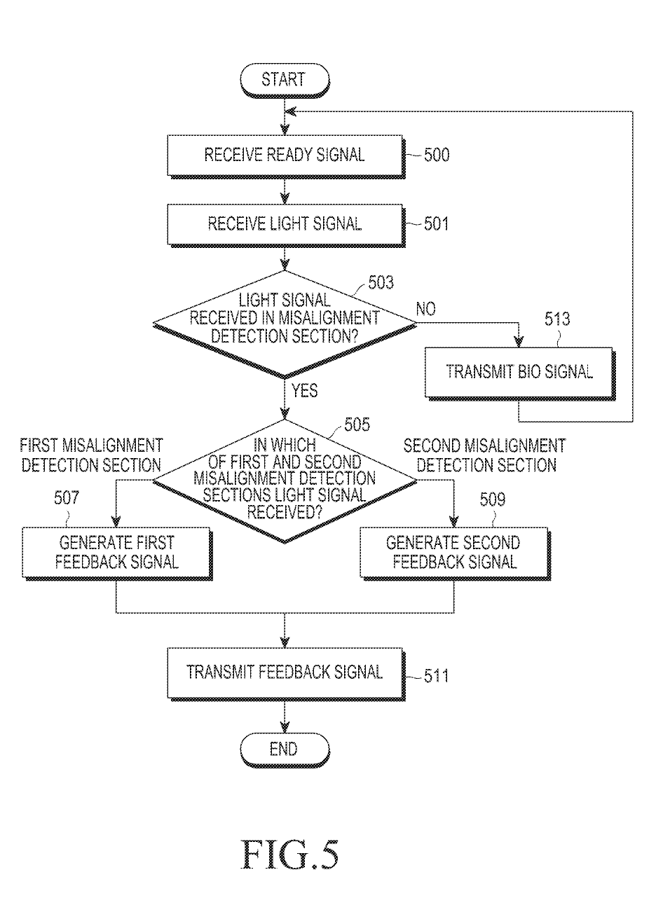

FIG. 5 is a flowchart illustrating a method for a bio sensor to detect misalignment with a bio signal receiving module according to an embodiment of the present disclosure.

Referring to FIG. 5, at operation 500, the bio sensor receives the ready signal from the receiving module.

At operation 501, the bio sensor receives the light signal from the receiving module. Thereafter, the bio sensor proceeds to operation 503.

At operation 503, the bio sensor determines whether the light signal is received in a misalignment detection section in which to detect misalignment between the bio sensor and the receiving module.

If the light signal is determined to be received in the misalignment detection section at operation 503, then the bio sensor proceeds to operation 505 at which the bio sensor determines in which of the first misalignment detection section to detect the receiving module being deviated to left of the bio sensor and the second misalignment detection section to detect the receiving module being deviated to right of the bio sensor the light signal is received at operation 505. For example, at operation 503, the bio sensor determines whether the misalignment between the receiving module and the bio sensor is a result of the receiving module being deviated to the left, or the receiving module being deviated to the right.

If the light signal is determined to be received in the first misalignment detection section at operation 505, then the bio sensor proceeds to operation 507 at which the bio sensor generates a first feedback signal that includes control information to indicate that the receiving module is deviated to the left of the bio sensor. Thereafter, the bio sensor proceeds to operation 511.

In contrast, if the light signal is determined to be received in the second misalignment detection section at operation 505, then the bio sensor proceeds to operation 509 at which the bio sensor generates a second feedback signal that includes control information to indicate that the receiving module is deviated to the right of the bio sensor. Thereafter, the bio sensor proceeds to operation 511.

At operation 511, the bio sensor transmits the first or second feedback signal to the receiving module.

In contrast, if the light signal is determined to have not been received in the misalignment detection section at operation 503, then bio sensor proceeds to operation 513 at which the bio sensor transmits bio signals.

In various embodiments such as, for example, the embodiment of FIG. 5, upon detection of misalignment with the receiving module (e.g., upon reception of the light signal in the misalignment detection section), the bio sensor generates and transmits the feedback signal to the receiving module. However, although not shown, upon reception of the light signal in the misalignment detection section, the bio sensor may directly proceed to operation 501 without transmitting any signal, thereby suppressing occurrence of interference due to transmission of a feedback signal.

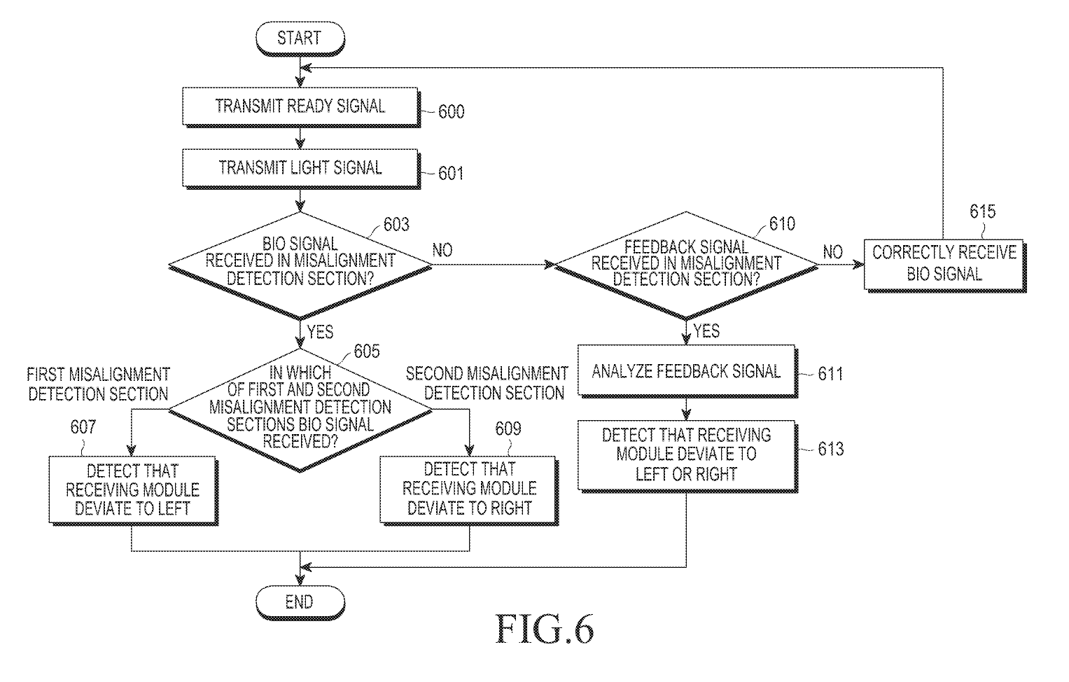

FIG. 6 is a flowchart illustrating a method for a bio signal receiving module to detect misalignment with a bio sensor according to an embodiment of the present disclosure.

Referring to FIG. 6, the bio signal receiving module (also referred to as a receiving module) transmits a ready signal to the bio sensor at operation 600.

At operation 601, the receiving module transmits the light signal to the bio sensor. Thereafter, the receiving module proceeds to operation 603.

At operation 603, the receiving module determines whether the bio signal from the bio sensor is received in the misalignment detection section in which to detect misalignment between the bio sensor and the receiving module.

If the bio signal is determined to be received in the misalignment detection section at operation 603, then the receiving module proceeds to operation 605 at which the receiving module determines in which of the first misalignment detection section to detect that the receiving module is deviated to left of the bio sensor and the second misalignment detection section to detect that the receiving module is deviated to right of the bio sensor the bio signal is received. For example, at operation 605, the receiving module determines whether the misalignment between the receiving module and the bio sensor is a result of the receiving module being deviated to the left, or the receiving module being deviated to the right.

If the bio signal is determined to be received in the first misalignment detection section at operation 605, then the receiving module proceeds to operation 607 at which the receiving module determines that the receiving module is deviated to the left of the bio sensor. According to various embodiments, the receiving module may be configured to provide an indication to a user or another device of the misalignment. According to various embodiments, the receiving module may correct alignment between the receiving module and the bio sensor. For example, the receiving module may autocorrect alignment between the receiving module and the bio sensor.

In contrast, if the bio signal is determined to be received in the second misalignment detection section at operation 605, then the receiving module proceeds to operation 609 at which the receiving module determines that the receiving module is deviated to the right of the bio sensor.

In contrast, if the bio signal is determined to have not been received in the misalignment detection section at operation 603, then the receiving module proceeds to operation 610 at which the receiving module determines whether a feedback signal is received in the misalignment detection section.

If the receiving module determines that a feedback signal is received at operation 610, then the receiving module proceeds to operation 611 at which the receiving module analyzes the feedback signal. Thereafter, the receiving module proceeds to operation 613.

At operation 613, the receiving module determines from the analysis result to which of the left and right of the bio sensor the receiving module is deviated. For example, the receiving module detects the direction in which the receiving module is deviated relative to the bio sensor.

In contrast, if the receiving module determines that no feedback signal is received in the misalignment detection section at operation 610, then the receiving module proceeds to operation 615 at which the receiving module determines that the receiving module is not deviated from the bio sensor (e.g., the receiving module determines that the receiving module is ready to correctly receive bio signals from the bio sensor). Thereafter, the receiving module proceeds to operation 600.

According to various embodiments, because the bio sensor is a passive device, signal transmission is enabled only by the transmitter, and thus, if the first and second misalignment detection sections of the receiving module are deviated off to an extent that they go out of coverage of the transmitter of the bio sensor, the misalignment between the receiving module and the bio sensor may not be detected despite the actual deviation. Thus, although not shown, in order to solve this problem, the receiving module may start a predetermined timer at a same time when the receiving module determines whether the bio signal is received in the misalignment detection section at operation 603, and if no signal has been received until expiration of the timer, the receiving module may determine that the receiving module and the bio sensor are misaligned.

According to various embodiments of the present disclosure, misalignment of a bio sensor and a bio sensor receiving module is detected and thus the problem of impossibility or error occurrence in transmitting or receiving bio signals due to the misalignment may be solved.

Various embodiments have thus been described, but it will be understood that various modifications can be made without departing the scope of the present disclosure. Thus, it will be apparent to those ordinary skilled in the art that the present disclosure is not limited to the various embodiments described, but can encompass not only the appended claims but the equivalents.

It will be appreciated that the various embodiments of the present disclosure may be implemented in a form of hardware, software, or a combination of hardware and software. The software may be stored as program instructions or computer readable codes executable on the processor on a non-transitory computer-readable medium. Examples of the non-transitory computer readable recording medium include magnetic storage media (e.g., Read Only Memories (ROMs), floppy disks, hard disks, and the like), and optical recording media (e.g., Compact Disk (CD)-ROMs, or Digital Versatile Discs (DVDs)). The non-transitory computer readable recording medium can also be distributed over network coupled computer systems so that the computer readable code is stored and executed in a distributed fashion. This media can be read by the computer, stored in the memory, and executed by the processor. Various embodiments may be implemented by a computer or portable terminal including a controller and a memory, and the memory may be an example of the computer readable recording medium suitable for storing a program or programs having instructions that implement the various embodiments of the present disclosure.

The present disclosure may be implemented by a program having codes for embodying the apparatus and method described in claims, the program being stored in a machine (or computer) readable storage medium. The program may be electronically carried on any medium, such as communication signals transferred via wired or wireless connection, and the present disclosure suitably includes its equivalent.

A user authentication agency may receive and store the program from a program provider connected thereto via cable or wirelessly. The program provider may include a memory for storing programs having instructions to perform the various embodiments of the present disclosure, information necessary for the various embodiments of the present disclosure, and the like, a communication unit for wired/wirelessly communicating with the mobile communication terminal 200, and a controller for sending the program to the mobile communication terminal 200 on request or automatically.

While the present disclosure has been shown and described with reference to various embodiments thereof, it will be understood by those skilled in the art that various changes in form and details may be made therein without departing from the spirit and scope of the present disclosure as defined by the appended claims and their equivalents.

What is claimed is:

1. A method for detecting, in a bio sensor, misalignment between the bio sensor and a bio signal receiving module, the method comprising:
receiving, by the bio sensor, a light signal from the bio signal receiving module;
determining, by the bio sensor, whether the light signal is received in a misalignment detection section;
determining, by the bio sensor, that the bio sensor and the bio signal receiving module are misaligned if the light signal is received in the misalignment detection section;
generating a feedback signal that comprises information to indicate misalignment between the bio sensor and the bio signal receiving module; and
transmitting, by the bio sensor, the feedback signal to the bio signal receiving module.

2. The method of claim 1, wherein the determining that the bio sensor and the bio signal receiving module are misaligned comprises determining a direction in which the bio signal receiving module is deviated from the bio sensor, and
wherein the feedback signal further comprises information to indicate the direction in which the bio signal receiving module is deviated from the bio sensor.

3. The method of claim 2, wherein the determining as to which direction the bio signal receiving module is deviated from the bio sensor comprises:
determining whether the light signal is received in a first misalignment detection section to detect the bio signal receiving module being deviated to the left of the bio sensor, or a second misalignment detection section to detect the bio signal receiving module being deviated to the right of the bio sensor;
determining that the bio signal receiving module is deviated to the left of the bio sensor if the light signal is received in the first misalignment detection section; and
determining that the bio signal receiving module is deviated to the right of the bio sensor if the light signal is received in the second misalignment detection section.

4. The method of claim 1, further comprising:
generating, by the bio sensor, a bio signal; and
transmitting, by the bio sensor, the bio signal to the bio signal receiving module, if the light signal is not received in the misalignment detection section.

5. The method of claim 1, further comprising:
transmitting, by the bio sensor, no bio signal if the light signal is received in the misalignment detection section.

6. A method for detecting, in a bio signal receiving module, misalignment between a bio sensor and the bio signal receiving module, the method comprising:
transmitting, by the bio signal receiving module, a light signal to the bio sensor;
determining, by the bio signal receiving module, whether a bio signal from the bio sensor is received in a misalignment detection section; and
determining, by the bio signal receiving module, that the bio signal receiving module and the bio sensor are misaligned if the bio signal is received in the misalignment detection section.

7. The method of claim 6, wherein the determining of whether the bio signal from the bio sensor is received in the misalignment detection section comprises:
determining whether the bio signal is received in a first misalignment detection section to detect the bio signal receiving module being deviated to the left of the bio sensor, or a second misalignment detection section to detect the bio signal receiving module being deviated to the right of the bio sensor, and
wherein the determining that the bio signal receiving module and the bio sensor are misaligned comprises:
determining that the bio signal receiving module is deviated to the left of the bio sensor if the bio signal is received in the first misalignment detection section; and
determining that the bio signal receiving module is deviated to the right of the bio sensor if the bio signal is received in the second misalignment detection section.

8. The method of claim 6, further comprising:
determining, if the bio signal is not received in the misalignment detection section, whether a feedback signal is received from the bio sensor, wherein the feedback signal comprises information to indicate misalignment between the bio sensor and the bio signal receiving module and information to indicate in which direction the bio signal receiving module is deviated from the bio sensor; and
analyzing, if the feedback signal is received, the feedback signal and determining whether the bio signal receiving module is deviated in a left direction or a right direction relative to the bio sensor.

9. The method of claim 8, further comprising:
determining, if the bio signal and the feedback signal are not received in the misalignment detection section, that the bio signal receiving module is not deviated relative to the bio sensor.

10. The method of claim 6, further comprising:
starting a timer at a same time when determining whether the bio signal from the bio sensor is received in the misalignment detection section, and determining that the bio signal receiving module and the bio sensor are misaligned if no bio signal has been received until expiration of the timer.

11. A bio sensor for detecting misalignment between the bio sensor and a bio signal receiving module, the bio sensor comprising:
a photovoltaic power source configured to receive a light signal from the bio signal receiving module;
a detector configured to determine whether the light signal is received in a misalignment detection section, and to determine that the bio sensor and the bio signal receiving module are misaligned if the light signal is received in the misalignment detection section; and
a transmitter configured to generate a feedback signal that comprises information to indicate misalignment between the bio sensor and the bio signal receiving module, and to transmit the feedback signal to the bio signal receiving module.

12. The bio sensor of claim 11, wherein the detector determines a direction in which the bio signal receiving module is deviated from the bio sensor, and
wherein the transmitter generates the feedback signal to include information to indicate the direction in which the bio signal receiving module is deviated from the bio sensor.

13. The bio sensor of claim 12, wherein the detector determines whether the light signal is received in a first misalignment detection section to detect the bio signal receiving module being deviated to the left of the bio sensor, or a second misalignment detection section to detect the bio signal receiving module being deviated to the right of the bio sensor, determines that the bio signal receiving module is deviated to the left of the bio sensor if the light signal is received in the first misalignment detection section, and determines that the bio signal receiving module is deviated to the right of the bio sensor if the light signal is received in the second misalignment detection section.

14. The bio sensor of claim 11, wherein the transmitter generates a bio signal, and transmits the bio signal to the bio signal receiving module, if the light signal is not received in the misalignment detection section.

15. The bio sensor of claim 11, wherein the transmitter transmits no bio signal if the light signal is received in the misalignment detection section.

16. A bio signal receiving module for detecting misalignment between a bio sensor and the bio signal receiving module, the bio signal receiving module comprising:
a transmitter configured to transmit a light signal to the bio sensor; and
a detector configured to determine whether a bio signal from the bio sensor is received in a misalignment detection section, and to determine that the bio signal receiving module and the bio sensor are misaligned if the bio signal is received in the misalignment detection section.

17. The bio signal receiving module of claim 16, wherein the detector determines whether the bio signal is received in a first misalignment detection section to detect the bio signal receiving module being deviated to the left of the bio sensor, or a second misalignment detection section to detect the bio signal receiving module being deviated to the right of the bio sensor, determines that the bio signal receiving module is deviated to the left of the bio sensor if the bio signal is received in the first misalignment detection section, and determines that the bio signal receiving module is deviated to the right of the bio sensor if the bio signal is received in the second misalignment detection section.

18. The bio signal receiving module of claim 16, wherein the detector determines, if the bio signal is not received in the misalignment detection section, whether a feedback signal is received from the bio sensor, wherein the feedback signal comprises information to indicate misalignment between the bio sensor and the bio signal receiving module and information to indicate in which direction the bio signal receiving module is deviated from the bio sensor, analyzes the feedback signal, and determines whether the bio signal receiving module is deviated in a left direction or right direction relative to the bio sensor.

19. The bio signal receiving module of claim 18, wherein the detector determines, if the bio signal and the feedback signal are not received in the misalignment detection section, that the bio signal receiving module is not deviated relative to the bio sensor.

20. The bio signal receiving module of claim 16, wherein the detector starts a timer at a same time when determining whether the bio signal from the bio sensor is received in the misalignment detection section, and determines that the bio signal receiving module and the bio sensor are misaligned if no bio signal has been received until expiration of the time
